(19) United States
(12) Patent Application Publication
(10) Pub. No.: US 2018/0252668 A1
(43) Pub. Date: Sep. 6, 2018

(54) ELECTROCHEMICAL CARBON DIOXIDE COMPRESSORS USING ANION EXCHANGE MEMBRANE

(71) Applicant: Xergy Inc., Georgetown, DE (US)

(72) Inventors: Bamdad Bahar, Georgetown, DE (US); Richard Sherrer, Landing, NJ (US)

(21) Appl. No.: 15/912,541

(22) Filed: Mar. 5, 2018

Related U.S. Application Data
(60) Provisional application No. 62/466,656, filed on Mar. 3, 2017.

(51) Int. Cl. G01N 27/407; G01N 27/404; G01N 27/416; G01N 27/30; G01N 33/00; G01N 27/406

(52) U.S. Cl. CPC G01N 27/4071; G01N 27/4045; G01N 27/4065; G01N 27/304; G01N 33/004; G01N 27/4162

(57) ABSTRACT

An electrochemical sensor for determining carbon dioxide gas concentration and electrochemical compressor for pumping carbon dioxide are constructed using an anion exchange membrane, catalyst film, and two electrodes. For the sensor, the membrane is compressed between the two electrodes and a voltage reading is output to a computer that then correlates the reading to a carbon dioxide concentration. For the compressor, the membrane is compressed between two plates and current is passed through the plates to pump carbon dioxide through.

### BACKGROUND OF THE INVENTION

### Cross Reference To Related Applications

The invention claims the benefit of priority to U.S. provisional patent application no, 6/466,656, filed on Mar. 3, 2017 and entitled ELECTROCHEMICAL CARBON DIOXIDE COMPRESSORS USING ANION EXCHANGE MEMBRANE; the entirety of which is hereby incorporated by reference herein.

### FIELD OF THE INVENTION

The invention is directed to electrochemical carbon dioxide compressor directly pumping carbon dioxide and to electrochemical carbon dioxide sensor directly measuring carbon dioxide with a unique anion exchange membrane

### BACKGROUND

Electrochemical compressors move gases at a controlled flow rate and pressure by moving the gases through a membrane via direct current. The amount of current applied to the electrochemical compressor is directly proportional to the amount of gas flowing through. These compressors are more efficient, more compact, and have tighter control than mechanical compressors.

Electrochemical sensors read the concentration level of a gas by measuring the potential difference between two or more electrodes created by a concentration gradient between the outside environment and the sensor's internal environment. This potential difference (in millivolts) is read by a meter and calibrated to a ppm level of the gas. These sensors are lower cost, more compact, and provide a more linear correlation between reading and gas concentration compared to current leading technology (i.e. infrared).

The compressors and sensors are constructed with electrodes, membrane, and catalyst as the working parts. Every marketed electrochemical sensor and hydrogen electrochemical compressors uses proton exchange membranes (PEMs) and a precious metal catalyst. PEMs allow for the exchange of protons (H”) across the membrane. Gas can react with water in PEM sensors to produce a signal by turning the water into protons and oxygen molecules.

PEM technology, however, cannot target carbon dioxide (CO2) to produce a voltage nor be electrolyzed to absorb voltage. CO2 sensors and compressors, therefore need different types of membrane to work. Anion exchange membranes (AEMs) interact with CO2 and water to induce a potential across the membrane.

AEMs allow for the exchange of anions across the membrane. In this case, the AEM moves bicarbonate anions across the membrane, creating a potential when the electrons are moved from one side of the membrane to the other. This allows for direct movement and sensing of CO2, of which there is no commercial electrochemical technology that can do this.

The compressor technology requires an influx of hydrogen gas to amplify the effect of carbon dioxide pumping. Hydrogen is a product of the cathode side of the electrochemical cell and a reactant of the anode side By pumping hydrogen into the system, the compressor has excess react to more readily push carbon dioxide through the compressor. Hydrogen is not overall consumed nor produced in the cell but having excess pushes the reaction toward the product side.

### SUMMARY OF THE INVENTION

The invention is directed to electrochemical carbon dioxide compressors, sensors and gas sensors using anion exchange membranes.

In an exemplary embodiment, an electrochemical sensor for the detection of carbon dioxide gas comprises an anion exchange membrane, film layer applied to said membrane and electrodes wired to output current to a reading device or to receive current from an external power source. An exemplary electrochemical sensor may also comprise a cavity for storing hydrating solution and a hydrophilic membrane separating said cavity and the electrodes for controlled water permeation. An exemplary film layer comprises of Platinum. The film layer may comprise a metal oxide, (MO₂) where M is Pt, Ir, Ru or Rh. An exemplary film layer may comprise an

A₂B₂O₇molecular structure, where A is Be, Mg, Ca, Sr, Ba, or a combination of said metals and where B is Pt, Ir, Ru, Rh, or a combination of said metals. The electrodes of an exemplary sensor may be are titanium, 316L stainless steel, or niobium. The hydrating solution may be distilled water or a sulfuric acid solution, for example. An exemplary hydrophilic membrane is perfluorosulfonic acid, such as Nafion, from Dupont.

An exemplary electrochemical compressor for the compression and movement of carbon dioxide gas comprises an anion exchange membrane, a film layer applied to said membrane and one or more gas diffusion layers, and one or more plates and current collectors. An exemplary electrochemical compressor comprises a catalytic transport fluid. The film layer may comprise Platinum, Pt or a metal oxide (MO₂), where M is Pt, Ir, Ru, or Rh. The film layer may comprise an A₂B₂O₇ molecular structure, wherein A is Be, Mg, Ca, Sr, Ba, or a combination of said metals and wherein B is Pt, Ir, Ru, Rh, or a combination of said metals. The gas diffusion layer may be or comprise carbon felt or titanium mesh. The plates of a exemplary compressor may comprise gold plated aluminum, 316 stainless steel, or titanium. The current collecting end plates may comprise gold plated aluminum, 316 stainless steel, or titanium. The catalytic transport fluid may be hydrogen. An exemplary compressor may be composed of multiple compressors stacked together.

The summary, of the invention is provided as a general introduction to some of the embodiments of the invention and is not intended to be limiting. Additional example embodiments including variations and alternative configurations of the invention are provided herein.

### BRIEF DESCRIPTION OF THE DRAWINGS

The accompanying drawings are included to provide a further understanding of the invention and are incorporated in and constitute a part of this specification, illustrate embodiments of the invention, and together with the description serve to explain the principles of the invention.

FIG. 1 shows a half-reaction happening on the cathode.

FIG. 2 shows a half-reaction happening on the anode.

FIG. 3 shows a diagram of an exemplary electrochemical sensor.

FIG. 4 shows a diagram of an exemplary electrochemical sensor.

FIG. 5 shows a diagram of an exemplary electrochemical compressor.

### DETAILED DESCRIPTION OF THE ILLUSTRATED EMBODIMENTS

Corresponding reference characters indicate corresponding parts throughout the several views of the figures. The figures represent an illustration of some of the embodiments of the present invention and are not to be construed as limiting the scope of the invention in any manner. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Certain exemplary embodiments of the present invention are described herein and are illustrated in the accompanying figures. The embodiments described are only for purposes of illustrating the present invention and should not be interpreted as limiting the scope of the invention. Other embodiments of the invention, and certain modifications, combinations and improvements of the described embodiments, will occur to those skilled in the art and all such alternate embodiments, combinations, modifications, improvements are within the scope of the present invention.

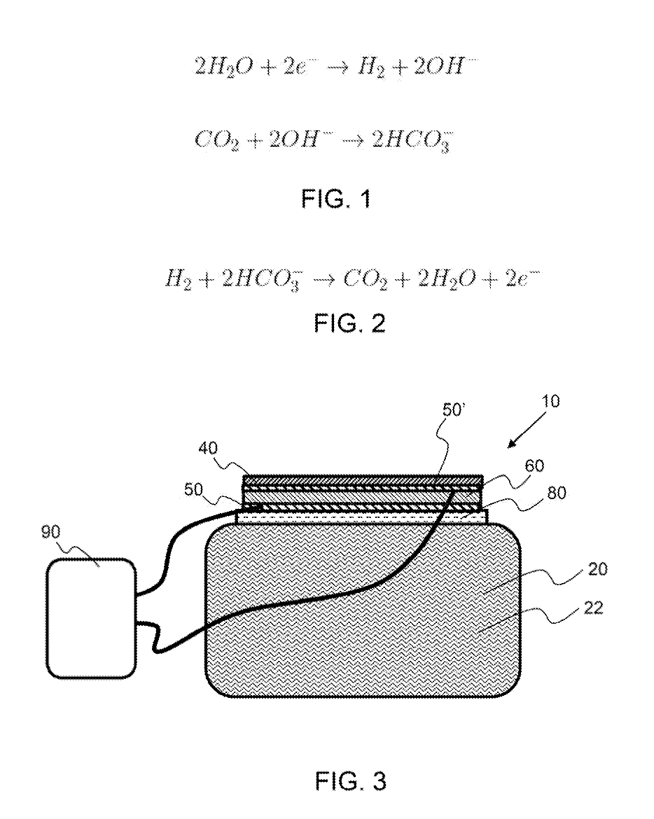

Referring now to FIG. 1. On the cathode side of the electrochemical cell, water decomposes into hydrogen and hydroxide ions. These hydroxide ions then combine with carbon dioxide molecules to form bicarbonate. By forming bicarbonate anions, the carbon dioxide now can interact with the AEM and moves across it.

FIG. 1 shows the half reaction at the cathode. The water molecule gets split into hydrogen gas and hydroxide anions. The carbon dioxide molecule in the water combines hydroxide anions to make bicarbonate anions that permeate across the membrane.

Referring now to FIG. 2. On the anode side, the bicarbonate ions combine with hydrogen gas to revert to carbon dioxide and water. Thus, completing the chemical reaction cycle.

FIG. 2 shows the half reaction at the anode. The bicarbonate combines with the hydrogen gas to revert back to carbon dioxide, water, and electrons.

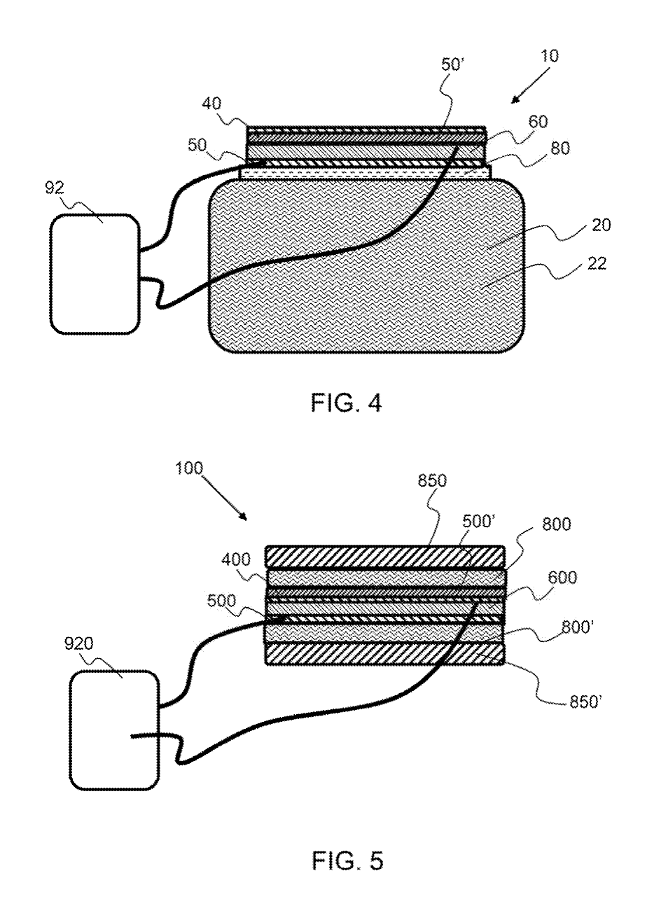

Referring to FIGS. 3 and 4, an exemplary electrochemical sensor 10 comprises a cavity 20 for storing a hydrating solution 22. Attached to an opening in the cavity is a hydrophilic membrane 80. A pair of electrodes 50, 50′ are coupled on the opposing side of the hydrophilic membrane from the cavity for storing a hydrating solution. An anion exchange membrane 60 is coupled to the electrodes and a film layer 40 is coupled to the anion exchange membrane. A pair of wires extend from the electrode to a current reading device 90. An external power source 92 may be coupled to the electrodes, as shown in FIG. 4.

As shown in FIG. 5 an exemplary electrochemical compressor 100, comprises a pair of electrodes 500, 500′ coupled to opposing sides of an anion exchange membrane 600. A film layer 400 is applied to the anion exchange membrane. Gas diffusion media 800, 800′ is configured on opposing sides of the n anion exchange membrane 600. Current collector 850, 850′ are configured outside of the gas diffusion media. A power source 920 may be coupled to the electrodes 500, 500′ to produce a voltage across the anion exchange membrane. A electrochemical fluid may be reacted on the electrodes.

One skilled in the art will also recognize, of course, that various changes, additions, or modifications of or to the methods described above may be made without substantively altering the compounds obtained or their characteristics. Such changes, additions, and modifications are therefore intended to be within the scope of the invention.

This written description uses examples to disclose the invention, including the best mode, and also to enable any person skilled in the art to practice the invention, including making and using any devices or systems and performing any related or incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

It will be apparent to those skilled in the art that various modifications, combinations and variations can be made in the present invention without departing from the spirit or scope of the invention. Specific embodiments, features and elements described herein may be modified, and/or combined in any suitable manner. Thus, it is intended that the present invention cover the modifications, combinations and variations of this invention provided they come within the scope of the appended claims and their equivalents.

What is claimed is:

1. An electrochemical sensor for the detection of carbon dioxide gas comprising of:
a) an anion exchange membrane;
b) a film layer applied to said membrane;
c) two electrodes wired to output current to a reading device or to receive current from an external power source;
d) a cavity for storing hydrating solution; and
e) a hydrophilic membrane separating said cavity and the electrodes for controlled water permeation.

2. The electrochemical sensor of claim 1 where the film layer is comprised of Pt.

3. The electrochemical sensor of claim 1 where the film layer is comprised of an MO₂metal oxide where M is Pt, lr, Ru, or Rh.

4. The electrochemical sensor of claim 1 where the film layer is comprised of an A₂B₂O₇ molecular structure.

5. The electrochemical sensor of claim 4, wherein A comprises Be, Mg, Ca, Sr, Ba, or a combination of said metals.

6. The electrochemical sensor of claim 4, wherein B comprises Pt, Ir, Ru, Rh, or a combination of said metals.

7. The electrochemical sensor of claim 1, wherein the electrodes are titanium, 316L stainless steel, or niobium.

8. The electrochemical sensor of claim 1, wherein the hydrating solution is distilled water or a sulfuric acid solution.

9. The electrochemical sensor of claim 1, wherein the hydrophilic membrane comprises perfluorosulfonic acid.

10. An electrochemical compressor for the compression and movement of carbon dioxide gas comprising a stack comprising:
a) an anion exchange membrane;
b) a film layer applied to said membrane;
c) two gas diffusion layers;
d) two plates;
e) two current collecting end plates; and
f) a catalytic transport fluid.

11. The electrochemical compressor of claim 10, wherein the film layer comprised of Pt.

12. The electrochemical compressor of claim 10, wherein the film layer is comprised of an MO₂ metal oxide where M is Pt, Ir, Ru, or Rh.

13. The electrochemical compressor of claim 10, wherein the film layer is comprised of an A₂B₂O₇ molecular structure.

14. The electrochemical compressor of claim 13, wherein A is Be, Mg, Ca, Sr, Ba, or a combination of said metals.

15. The electrochemical compressor of claim 13, wherein B is Pt, Ir, Ru, Rh, or a combination of said metals.

16. The compressor of claim 10 where the gas diffusion layer is carbon felt or titanium mesh.

17. The electrochemical compressor of claim 10, wherein the plates are gold plated aluminum, 316 stainless steel, or titanium.

18. The electrochemical compressor of claim 10, wherein the current collecting end plates are gold plated aluminum, 316 stainless steel, or titanium

19. The electrochemical compressor of claim 10, wherein the catalytic transport fluid is hydrogen.

20. The electrochemical compressor of claim 10, comprising two or more stacks.
